(19) United States
(12) Patent Application Publication
(10) Pub. No.: US 2002/0055638 A1
(43) Pub. Date: May 9, 2002

(54) Process for the preparation of carboxamides by oxidation of aldehydes in the presence of amines

(75) Inventors: Thomas Riermeier, Floersheim (DE); Matthias Beller, Rostock (DE); Annegret Tillack, Rostock (DE); Ivo Rudloff, Southampton (GB)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: Degussa AG, Duesseldorf (DE)

(21) Appl. No.: 09/927,235

(22) Filed: Aug. 13, 2001

(30) Foreign Application Priority Data
Aug. 11, 2000 (DE) 100 39 247.4

(51) Int. Cl. C 07D 4 1/02; C07D 211/60

(52) U.S. Cl. 546/189; 546/225; 548/530; 560/15; 564/152

(57) ABSTRACT

A mono-, bi- and/or polyfunctional amide of the formulae (Ia) and/or (Ib),

R¹—CO—NR²R³ (Ia)

R⁴R⁵N—CO—R⁶—CO—NR²R³ (Ib)

is prepared by reacting an aldehyde and an amine in the presence of a transition metal catalyst and an oxidizing agent.

### BACKGROUND OF THE INVENTION

1. Field of the Invention

The present invention relates to a process for the preparation of aliphatic and aromatic carboxamides from aldehydes and amines in the presence of a transition metal catalyst and an oxidizing agent.

2. Description of the Background

Carboxamides have industrial significance in the preparation of fine chemicals in that they are starting materials for polymers (polyamides), functional materials, agrochemicals and pharmaceutically active ingredients.

In general, carboxamides are prepared by reacting activated carboxylic acid derivatives, such as, for example, carboxylic acid esters, carboxylic acid anhydrides, carboxylic acid halides, or the like with amines in the presence of a catalyst or a condensation reagent (D. Döpp, H. Döpp, in Houben-Weyl, Methoden der Organischen Chemie [Methods of organic chemistry], Volume E5; J. Falbe, Ed.; Georg Thieme Verlag: Stuttgart, 1985, p. 934. (b) in Comprehensive Organic Transformations: a guide to functional group preparations; R. C. Larock, Ed.; VCH: Weinheim, 1989, p. 885, 994).

In these processes of preparation, stoichiometric amounts of by-products, notably salt wastes, are formed. Known amide syntheses, which avoid the ecological problems associated with by-product production, are heterogeneously catalyzed aminations of carboxylic acids. However, these reactions proceed under drastic reaction conditions of very high reaction temperature, meaning that carboxamides with sensitive functional groups cannot be synthesized selectively (Beckwitch in Zabicky, The Chemistry of Amides, Wiley, NY, 1970, 105-109).

Another problem of amide syntheses is sometimes the accessibility of the corresponding carboxylic acid derivative.

An alternative amide synthesis route starts from inexpensive aldehydes (Y. Tamura, Y. Yamada, Z. Yoshida, Synthesis 1983, 474). Tamura and coworkers thus describe the synthesis of amides from aldehydes in the presence of palladium catalysts. Aryl bromides are used as oxidizing agent. This process has the disadvantage that stoichiometric amounts of expensive bromobenzene have to be used and stoichiometric quantities of bromine waste are formed.

Other known processes of preparing amides from aldehydes (K. Nakagawa, H. Onoue, K. Minami, Chem. Commun. 1966, 4319. S. Fukuoka, M. Ryang, S. Tsutsumi, J. Org. Chem. 1971, 36, 2721) require stoichiometric amounts of transition metal compounds, thus rendering these processes both economically and also ecologically unattractive. Naota describes a ruthenium-catalyzed variant of this reaction (Naota, Synlett, 1991, 693). In this process, benzylidene acetone, which is not readily accessible and can be recycled only at very high cost, is used as oxidizing agent. In addition, DE 2953007 C1 describes the use of heterogeneous catalysts for this reaction, in which case the yields achieved are low and do not satisfy industrial requirements.

In view of the above discussion, it is clear that there is a continuing need for a process by which amides can be readily prepared from aldehydes and amines in a manner which is as simple as possible, cost-effective and can be employed on an industrial scale.

### SUMMARY OF THE INVENTION

Accordingly, one object of the present invention is to provide a method of preparing amides by the reaction of an aldehyde and an amine which is simple and cost effective.

Briefly, this object and other objects of the present invention as hereinafter will become more readily apparent can be attained by a process of preparing mono-, bi- and/or polyfunctional amides of formulae (Ia) and/or (Ib),

R¹_13 CO—NR²R³ (Ia)

R⁴R⁵N—CO—R⁶—CO—NR²R³ (Ib)

wherein R¹ is a (C₁-C₁₈)-alkyl radical, a (C₂-C₁₈)-alkenyl radical or a (C₂-C₁₈)-alkynyl radical, each of which may be branched, linear or cyclic, hydrogen or an aromatic aryl radical or a heteroaryl radical, each of which contains up to 14 carbon atoms and, wherein, in the case of the heteroaryl radical, one to four heteroatoms selected from the group consisting of N, O and S;

where the alkyl radical, the alkenyl radical, the alkynyl radical and/or the aryl radical may carry, in addition to hydrogen atoms and the amide group, up to five substituents which, independently of one another, are (C₁-C₈)-alkyl, O-alkyl-(C₁-C₈), O-aryl, OCO-alkyl-(C₁-C₈), OCO-aryl, O-phenyl, phenyl, aryl, fluorine, chlorine, bromine, iodine, OH, NO₂, Sialkyl₃-(C₁-C₈), CN, COOH, SO₃H, NH-alkyl-(C₁-C₈), NH-aryl, N-alkyl₂-(C₁-C₈), N-aryl₂, SO₂-alkyl-(C₁-C₆), SO₂-aryl, SO-alkyl-(C₁-C₆), CF₃, NHCO-alkyl-(C₁-C₄), COO-alkyl-(C₁-C₈), COOaryl, CONH₂, CO-alkyl-(C₁-C₈), CO-aryl, NHCOH, NHCOO-alkyl-(C₁-C₄), CO-phenyl, COO-phenyl, CHCH—CO₂-alkyl-(C₁-C₈), PO-phenyl₂, POalkyl₂-(C₁-C₄), PO₃H₂, PO(O-alkyl-(C₁-C₆))₂ and SO₃-alkyl-(C₁-C₄);

where the aryl radical is a five-, six-, or seven-membered aromatic or heteroaromatic ring containing one to four heteroatoms selected from the group consisting of N, O and S and where aromatic, heteroaromatic and/or aliphatic rings having 4 to 16 carbon atoms can be fused to the aryl ring, where 1 to 8 carbon atoms may be replaced by heteroatoms selected from the group consisting of N, O and S, and in which

R² to R⁵, independently of one another, are hydrogen, alkyl-(C₁-C₁₈), aryl, where alkyl and aryl have the meanings given above and may have up to 5 of the above-mentioned substituents, in addition to hydrogen, and

in which R⁶ is a (C₁-C₁₈)-alkylene radical, which may be branched, linear and/or cyclic, or is an aromatic arylene radical containing up to 14 carbon atoms, where the alkylene radical and/or the arylene radical can carry, in addition to hydrogen atoms and the amide groups, up to five substituents which, independently of one another, are (C₁-C₈)-alkyl, O-alkyl-(C₁-C₈), O-aryl, OCO-alkyl-(C₁-C₈), OCO-aryl, O-phenyl, phenyl, aryl, fluorine, chlorine, bromine, iodine, OH, NO₂, Sialkyl₃-(C₁-C₈), CN, COOH, SO₃H, NH-alkyl-(C₁-C₈), NH-aryl, N-alkyl₂-(C₁-C₈), N-aryl₂, SO₂-alkyl-(C₁-C₆), SO₂-aryl, SO-alkyl-(C₁-C₆), CF₃, NHCO-alkyl-(C₁-C₄), COO-alkyl-(C₁-C₈), COOaryl, CONH₂, CO-alkyl-(C₁-C₈), CO-aryl, NHCOH, NHCOO-alkyl-(C₁-C₄), CO-phenyl, COO-phenyl, CHCH-CO₂-alkyl-(C₁-C₈), PO-phenyl₂, POalkyl₂-(C₁-C₄), PO₃H₂, PO(O-alkyl-(C₁-C₆))₂, SO₃-alkyl-(C₁-C₄), where the aryl radical is also a five-, six- or seven-membered aromatic or heteroaromatic ring, where the heteroaromatic ring may contain one to four heteroatoms selected from the group consisting of N, O and S, where an aromatic, heteroaromatic and/or aliphatic ring having 4 to 16 carbon atoms may be fused to this ring, where 1 to 8 carbon atoms of these ring systems may be replaced by heteroatoms selected from the group consisting of N, O and S,

by reacting an aldehyde and/or a dialdehyde of formula (IIa) and/or (IIb)

R¹—CHO (Iia)

OHC—R⁶—CHO (Iib)

with an amine of the formula (IIIa,b),

R⁴R⁵NH (IIIa)

HNR²R³ (IIIb)

wherein R¹ to R⁶ have the meanings stated above for formulae (Ia) and (Ib), in the presence of a transition metal catalyst selected from a Group VIII metal and an oxidizing agent.

### DETAILED DESCRIPTION OF THE PREFERRED EMBODIMENTS

In the present invention, a particularly preferred aspect is the preparation of alkylamides, preferably dialkylamnides, of formula (I), in which the radicals R² to R⁵ are (C₁-C₈)-alkyl or the substituted (C₁-C₈)-alkyls.

The process of the invention has proven particularly useful for the preparation of substituted benzamides, fatty amides and lower aliphatic amides.

Suitable solvents for the process include generally inert organic solvents. Aliphatic ethers, aromatic and/or aliphatic hydrocarbons and esters, and mixtures thereof are particularly suitable. In addition, the reaction can also be conducted in alcohols, water, amines or without solvents.

The reaction preferably proceeds at temperatures ranging from 20 to 200° C.; in many cases, it has proven useful to work at temperatures ranging from 60 to 180° C., preferably 80 to 140° C. The reaction can be conducted under pressure, particularly if low-boiling aldehydes or amines are used.

In the reactions, it is frequently advantageous to add a base as a cocatalyst to the reaction mixture. Suitable for this purpose are trialkylamines, which may be alicyclic or open-chain, or alkali metal or alkaline earth metal salts of aliphatic or aromatic carboxylic acids, such as acetates, propionates, benzoates or the corresponding carbonates, hydrogencarbonates, phosphates, hydrogenphosphates or hydroxides, preferably of lithium, sodium, potassium, calcium, magnesium, cesium or mixtures of such bases.

Suitable transition metal catalysts include metal compounds of Group VIII, such as Rh, Pd, Ir, Ru, Co, Pt, and the like. Preferred transition metal catalysts are Rh, Ir, Pt and Ru. Particularly preferred are rhodium catalysts. The catalysts can be used as homogeneous metal complexes or as heterogeneous catalysts.

Examples of transition metal catalysts which may be used include: bis(1,5-cyclooctadiene)rhodium(I) tetrafluoroborate, (1,5-cyclooctadiene)rhodium(I) acetylacetonate, dimeric (1c,5c-cyclooctadiene)rhodium(I) chloride, palladium(II) acetate, palladium(II) chloride, lithium tetrachloropalladate, palladium(II) acetylacetonate, bisacetonitrile palladium(II) chloride, bis(1,5-cyclooctadiene)-iridium(I) tetrafluoroborate, (1,5-cyclooctadiene)iridium(I) acetylacetonate, dimeric (1c,5c-cyclooctadiene)iridium(I) chloride, 1c,5c-cyclooctadiene platinum(II) chloride, platinum(II) acetylacetonate, carbonyltris(triphenylphosphine)ruthenium(II) dihydride and ruthenium(II) acetylacetonate.

In the process of the invention, the amount of catalyst employed normally ranges from 0.001 mol. %-10 mol. %. Preferably, 0.01 to 5 mol. % of catalyst is used.

In order to scavenge the hydrogen liberated during the reaction, it is necessary, in order to achieve high yields, to add an oxidizing agent. Suitable oxidizing agents include oxygen-containing oxidizing agents, such as N-oxides or peroxides, hypochlorite, oxygen and air. N-oxides, hydrogen peroxide and alkyl peroxides are particularly suitable.

The process of the invention can not only be conducted easily, but produces amides in high yield with high catalyst productivity and purity. Moreover, the oxidizing agents which are used can be readily recycled. As a result, the process described is particularly suitable for amide synthesis on an industrial scale.

The amides prepared of the invention can be used, inter alia, as intermediates for pharmaceuticals and agrochemicals, as building blocks for polymers and materials.

Having now generally described this invention, a further understanding can be obtained by reference to certain specific Examples which are provided herein for purposes of illustration only and are not intended to be limiting unless otherwise specified.

General procedure

A 0.01 to 5 mol. % amount of the transition metal catalyst, 0.22 mmol of base and 0.44 to 2.2 mmol of the oxidizing agent are suspended in 5 ml of solvent. At room temperature, 2.2 to 6.6 mmol of amine and 2.2 to 6.6 mmol of aldehyde are added thereto. The reaction mixture is heated at 80 to 140° C. for 8 to 20 hours with stirring in a pressure tube. The yield is determined by gas chromatography using hexadecane as internal standard.

The solvent is removed under reduced pressure, and the residue is purified by column chromatography.

### EXAMPLES

### Example 1

A 0.0223 g amount of [Rh(COD)₂]BF₄, 0.0304 g of potassium carbonate and 0.258 g of N-methylmorpholine N-oxide are suspended in 5 ml of toluene. At room temperature, 0.19 ml of morpholine and 0.45 ml of benzaldehyde, and 0.05 ml of hexadecane as internal standard are added thereto. The reaction mixture is heated at 140° C. for 8 hours with stirring in a pressure tube. The mixture is analyzed using gas chromatography (GC). A 0.41 g amount of 4-benzoylmorpholine is found. This amount corresponds to a yield of 100% with respect to the morpholine used.

### Example 2

A 0.0136 g amount of [Rh(COD)Cl]₂, 0.0304 g of potassium carbonate and 0.258 g of N-methylmorpholine N-oxide are suspended in 5 ml of toluene. At room temperature, 0.43 ml of piperidine and 0.225 ml of benzaldehyde, and 0.05 ml of hexadecane as internal standard are added thereto. The reaction mixture is heated at 140° C. for 20 hours with stirring in a pressure tube. The mixture is analyzed by gas chromatography (GC). A 0.39 g amount of 1-benzoylpiperidine is found. This amopunt corresponds to a yield of 93% with respect to the benzaldehyde used.

### Example 3

A 0.0223 g amount of [Rh(COD)₂]BF₄, 0.0304 g of potassium carbonate and 0.258 g of N-methylmorpholine N-oxide are suspended in 5 ml of TBF. At room temperature, 0.26 ml of N-methylbutylamine and 0.45 ml of benzaldehyde, and 0.05 ml of hexadecane as internal standard are added thereto. The reaction mixture is heated at 100° C. for 20 hours with stirring in a pressure tube. The mixture is analyzed by gas chromatography (GC). A 0.39 g amount of N-benzoyl-N-methylbutylamine is found. This amount corresponds to a yield of 94% with respect to the N-methylbutylamine used.

Additional Examples

The reactions described in Table 1 were conducted as follows:

A 0.0223 g amount of [Rh(COD)₂]BF₄, 0.0304 g of potassium carbonate and 0.258 g of N-methylmorpholine N-oxide are suspended in 5 ml of the given solvent. At room temperature, 2.2 mmol of the specified amine and 4.4 mmol of the specified aldehyde (or of the molar ratio of amine to aldehyde specified in each case), and 0.05 ml of hexadecane as internal standard are added thereto. The reaction mixture is heated at the specified temperature for the given time with stirring in a pressure tube. The mixture is analyzed by gas chromatography (GC). The yields obtained are based on the reactants used in substoichiometric amounts in each case.

| | | Aldehyde: | | | | |
|---|---|---|---|---|---|---|
| | | amine | | Temp. | Temp. | Yield of |
| No. | Reactants | molar ratio | Solv. | [° C.] | [h] | amide [%] |
| 1 2 3 | 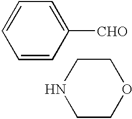 | 2:1 2:1 1.2:1 | THF Toluene Toluene | 100 140 140 | 8 8 8 | 100 100 88 |
| 4 5 | 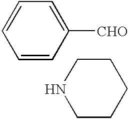 | 2:1 1:2 | Toluene Toluene | 140 140 | 20 8 | 89 83 |
| 6 7 | 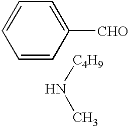 | 2:1 1:2 | Toluene THF | 120 100 | 20 20 | 69 63 |
| 8 9 | 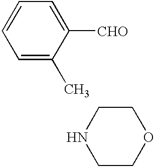 | 2:1 1:2 | Toluene Toluene | 140 140 | 8 8 | 100 82 |
| 10 11 | 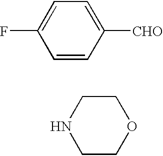 | 2:1 1:2 | THF THF | 100 100 | 20 8 | 94 56 |
| 12 13 | 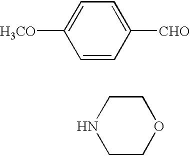 | 2:1 1:3 | Toluene Toluene | 140 140 | 8 8 | 57 84 |
| 14 15 | 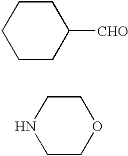 | 2:1 1:2 | THF THF | 100 100 | 8 8 | 48 58 |
| 16 17 | 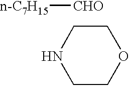 | 2:1 1:3 | Toluene Toluene | 140 140 | 8 8 | 29 61 |

The disclosure of German priority Application No. 100 39 247.4 filed August I 1, 2000 is hereby incorporated by reference into the present application.

Obviously, numerous modifications and variations of the present invention are possible in light of the above teachings. It is, therefore, to be understood that within the scope of the appended claims, the invention may be practiced otherwise than as specifically described herein.

What is Claimed as New and is Intended to be Secured by Letters Patent is:

1. A process for the preparation of mono-, bi- and/or polyflinctional amides of formulae (Ia) and/or (Ib),
R¹—CO—NR²R³ (Ia)
R⁴R⁵N—CO—R⁶—CO—NR²R³ (Ib)
wherein R¹ is a (C₁-C₁₈)-alkyl radical, a (C₂-C₁₈)-alkenyl radical or a (C₂-C₁₈)-alkynyl radical, each of which may be branched, linear or cyclic, hydrogen or an aromatic aryl radical or a heteroaryl radical, each of which contains up to 14 carbon atoms and wherein the heteroaryl radical contains one to four heteroatoms selected from the group consisting of N, O and S, where each of these radicals optionally carries, in addition to hydrogen atoms and the amide group, up to five substituents which, independently of one another, are (C₁-C₈)-alkyl, O-alkyl-(C₁-C₈), O-aryl, OCO-alkyl-(C₁-C₈), OCO-aryl, O-phenyl, phenyl, aryl, fluorine, chlorine, bromine, iodine, OH, NO₂, Sialkyl₃-(C₁-C₈), CN, COOH, SO₃H, NH-alkyl-(C₁-C₈), NH-aryl, N-alkyl₂-(C₁-C₈), N-aryl₂, SO₂-alkyl-(C₁-C₆), SO₂-aryl, SO-alkyl-(C₁-C₆), CF₃, NHCO-alkyl-(C₁-C₄), COO-alkyl-(C₁-C₈), COOaryl, CONH₂, CO-alkyl-(C₁-C₈), CO-aryl, NHCOH, NHCOO-alkyl-(C₁-C₄), CO-phenyl, COO-phenyl, CHCH-CO₂-alkyl-(C₁-C₈), PO-phenyl₂, POalkyl₂-(C₁-C₄), PO₃H₂, PO(O-alkyl-(C₁-C₆))₂, SO₃-alkyl-(C₁-C₄), where the aryl radical is a five-, six-, or seven-membered hydrocarbyl aromatic ring or a heteroaromatic ring containing one to four heteroatoms selected from the group consisting of N, O and S, wherein an aromatic, heteroaromatic and/or aliphatic ring having 4 to 16 carbon atoms and in which optionally 1 to 8 carbon atoms of the ring are replaced by heteroatoms selected from the group consisting of N, O and S, optionally is fused to the ring;
R² to R⁵, independently of one another, are hydrogen, (C₁-C₁₈)-alkyl, aryl, where alkyl and aryl have the meanings stated above and optionally has up to 5 substituents as defined above, in addition to hydrogen, and in which
R⁶ is a (C₁-C₁₈)-alkylene radical, which may be branched, linear and/or cyclic, or is an aromatic arylene radical containing up to 14 carbon atoms,
wherein the alkylene radical and/or the arylene radical optionally carries, in addition to hydrogen atoms and the amide groups, up to five substituents which, independently of one another, are (C₁-C₈)-alkyl, O-alkyl-(C₁-C₈), O-aryl, OCO-alkyl-(C₁-C₈), OCO-aryl, O-phenyl, phenyl, aryl, fluorine, chlorine, bromine, iodine, OH, NO₂, Sialkyl₃-(C₁-C₈), CN, COOH, SO₃H, NH-alkyl-(C₁-C₈), NH-aryl, N-alkyl₂-(C₁-C₈), N-aryl₂, SO₂-alkyl-(C₁-C₆), SO₂-aryl, SO-alkyl-(C₁-C₆), CF₃, NHCO-alkyl-(C₁-C₄), COO-alkyl-(C₁-C₈), COOaryl, CONH₂, CO-alkyl-(C₁-C₈), CO-aryl, NHCOH, NHCOO-alkyl-(C₁-C₄), CO-phenyl, COO-phenyl, CHCH-CO₂-alkyl-(C₁-C₈), PO-phenyl₂, POalkyl₂-(C₁-C₄), PO₃H₂, PO(O-alkyl-(C₁-C₆))₂, SO₃-alkyl-(C₁-C₄), where the aryl radical is also a five-, six- or seven-membered hydrocarbyl aromatic ring or a heteroaromatic ring, where the heteroaromatic ring optionally contains one to four heteroatoms selected from the group consisting of N, O and S, wherein a hydrocarbyl aromatic, heteroaromatic and/or aliphatic ring having 4 to 16 carbon atoms and optionally having 1 to 8 carbon atoms replaced by heteroatoms selected from the group consisting of N, O and S, optionally is fused to the ring, comprising:
reacting an aldehyde and/or a dialdehyde of formula (IIa) and/or (IIb)
R¹—CHO (Iia)
OHC—R⁶—CHO (Iib)
with an amine of formula (IIIa,b),
R⁴R⁵NH (IIIa)
HNR²R³ (IIIb)
wherein R¹ to R⁶ have the meanings stated above, in the presence of a transition metal catalyst of Group VIII and an oxidizing agent.

2. The process as claimed in claim 1, wherein the radicals R² to R⁵ are (C₁-C₈)-alkyl or substituted (C₁-C₈)-alkyl.

3. The process as claimed in claim 1, wherein the transition metal catalyst is a metal compound containing Rh, Pd, fIr, Ru, Co or Pt.

4. The process as claimed in claim 1, wherein said catalyst is a rhodium catalyst.

5. The process as claimed in claim 1, wherein the catalyst is bis( 1,5-cyclooctadiene)rhodium(I) tetrafluoroborate, (1,5-cyclooctadiene)rhodium(I) acetylacetonate, dimeric (1c,5c-cyclooctadiene)rhodium(l) chloride, palladium(II) acetate, palladium(II) chloride, lithium tetrachloropalladate, palladium(II) acetylacetonate, bisacetonitrile palladium(II) chloride, bis(1,5-cyclooctadiene)-iridium(I) tetrafluoroborate, (1,5 -cyclooctadiene)iridium(I) acetylacetonate, dimeric (1c,5c-cyclooctadiene)iridium(I) chloride, 1c,5c-cyclooctadiene platinum(II) chloride, platinum(II) acetylacetonate, carbonyltris(triphenyl-phosphine)ruthenium(II) dihydride or ruthenium(II) acetylacetonate.

6. The process as claimed in claim 1, wherein amount of catalyst ranges from 0.001 to 10 mol. %.

7. The process as claimed in claim 1, wherein the oxidizing agent is an oxygen-containing oxidizing agent.

8. The process as claimed in claim 1, wherein the oxidizing agent is an N-oxide, a peroxide, a hypochlorite, oxygen or air.

9. The process as claimed in claim 1, wherein the oxidizing agent is hydrogen peroxide or an alkyl peroxide.

10. The process as claimed in claim 1, wherein the reaction is conducted in the presence of a base as a cocatalyst in the reaction mixture.

11. The process as claimed in claim 1, wherein the reaction is conducted at a temperature ranging from 20 to 200° C.

12. The process as claimed in claim 11, wherein the reaction is conducted at a temperature ranging from 60 to 180° C.

13. The process as claimed in claim 12, wherein the reaction is conducted at a temperature ranging from 80 to 140° C.

14. The process as claimed in claim 10, wherein the base cocatalyst is an alicyclic or open-chain trialkylamine, or an alkali metal or alkaline earth metal salt of an aliphatic or aromatic carboxylic acid.

15. The process as claimed in claim 14, wherein the alkali metal or alkaline earth metal salt of an aliphatic or aromatic carboxylic acid is a lithium, sodium, potassium, calcium, magnesium or cesium acetate, propionate or benzoate or the corresponding carbonate, hydrogencarbonate, phosphate, hydrogenphosphate or hydroxide.

16. A method of preparing a pharmaceutical or an agrochemical, comprising:
in a step of production of a pharmaceutical or agrochemical which requires a mono-, bi- or polyfunctional amide as an intermediate reactant, conducting said step with the mono-, bi- or polyfunctional amide prepared by the process of claim 1.
